(19) United States
(12) Patent Application Publication
(10) Pub. No.: US 2010/0203614 A1
(43) Pub. Date: Aug. 12, 2010

(54) FATTY ALDEHYDE REDUCTASE

(75) Inventors: Brad Wahlen, Hyrum, UT (US); Brett Barney, Minneapolis, MN (US); Lance Seefeldt, Providence, UT (US)

Correspondence Address: UTAH STATE UNIVERSITY;TECHNOLOGY COMMERCIALIZATION OFFICE, 570 RESEARCH PARK WAY, SUITE 101, NORTH LOGAN, UT, 84341, US

(73) Assignee: Utah State University, North Logan, UT (US)

(21) Appl. No.: 12/704,801

(22) Filed: Feb. 12, 2010

Related U.S. Application Data
(60) Provisional application No. 61/151,957, filed on Feb. 12, 2009.

(51) Int. Cl. C12N 9/02; C12N 15/00

(52) U.S. Cl. 435/189; 536/23.2

(57) ABSTRACT

This disclosure relates to the polynucleotide sequences of SEQ ID NO: 1 and SEQ ID NO: 4, and to the amino acid sequences of SEQ ID NO: 2 and SEQ ID NO: 3, and to novel fatty aldehyde reductase enzymes provided by the same.

### CROSS-REFERENCE TO RELATED APPLICATIONS

This application claims the benefit of U.S. Provisional Application No. 61/151,957, filed on Feb. 12, 2009, and entitled “Fatty Aldehyde Reductase” which is incorporated by reference in its entirety herein.

### REFERENCE TO SEQUENCE LISTING

This application contains one or more sequence listings in paper and computer readable form; the information recorded in computer readable form is identical to the written sequence listing.

### FIELD OF DISCLOSURE

This disclosure is in the field of biotechnology.

### BACKGROUND OF THE INVENTION

Wax esters are a family of compounds with a basic structure composed of long chain fatty acids linked to long chain alcohols. The unique properties of wax esters make them valuable as additives in cosmetic and medical formulations, as well as high grade lubricants and food additives. Certain microbes have been shown to produce and accumulate wax esters, probably as energy storage compounds. A four enzyme pathway has been proposed for bacterial wax ester synthesis and is disclosed herein in FIG. 1.

Referring now to FIG. 1, the first step in the pathway involves the formation of fatty acyl CoA from a fatty acid by the action of a fatty acid: CoA ligase that also utilizes CoA and MgATP. Next, the fatty acyl CoA is reduced to the corresponding fatty aldehyde by the NADPH-dependent acyl-CoA reductase. It has been proposed that the fatty aldehyde might then be reduced to a fatty alcohol by a fatty aldehyde reductase, however, to the best of applicants' knowledge there have been no fatty aldehyde reductase enzymes indentified, isolated and characterized from any bacterial source; therefore, the proposed fatty aldehyde reductase is represented in FIG. 1 as “unknown.” The final step in wax ester formation is catalyzed by a wax ester synthase; the enzyme wax ester synthase/acyl coenzyme A: diacylglycerol acyltransferase (WS/DGAT). WS/DGAT has been purified from bacteria and partially characterized.

WS/DGAT shows a broad substrate range for alcohols ranging from ethanol to triacontanol and for acyl-CoAs of various lengths. Branched and aromatic alcohols also serve as substrates for WS/DGAT. The wide substrate range of WS/DGAT offers the possibility of producing a number of wax esters biologically.

The gene CER4 from the plant Arabidopsis thaliana is proposed to code for a wax ester biosynthetic enzyme. When CER 4 gene was disrupted in Arabidopsis, a phenotype resulted with significant decreases in concentration of measured primary alcohols and wax esters and slightly elevated levels of aldehydes found in the waxy cuticle that coats the aerial surfaces of the plant. The CER4 gene is tentatively assigned as a fatty aldehyde reductase. To the best of applicants' knowledge, no fatty aldehyde reductase of bacterial origin has ever been isolated or characterized.

Whole genome sequences for several bacteria with WS/DGAT genes are known. One such bacterium is Marinobacter aquaeolei VT8 (accession NC_{—}008740.1). Genbank accession numbers NC_{—}008740.1, NC_{—}008740.1:2484020 . . . 2485561, and YP_{—}959486 describe proposed nucleotide sequences and amino acid sequences, including a hypothetical protein.

### BRIEF SUMMARY OF THE INVENTION

### Definitions

As used herein, “substantial identity” or “substantially identical to” indicates that a polynucleotide or amino acid sequence has a greater degree of identity with the embodiments of the invention described herein than with prior art polynucleotide or amino acid sequences.

As used herein, “polypeptide” means an amino acid sequence containing at least 10 to more than 100 amino acids, such that the length of the amino acid sequence is not limited by any arbitrary upper boundary, and includes both naturally and non-naturally occurring peptides and proteins. The term “polypeptide” is also meant to include the amino acid sequence in any one or more structural forms commonly referred to as primary protein structures, secondary protein structures, tertiary protein structures, or quaternary protein structures. For clarity's sake the phrase “polypeptide or protein” is sometimes used and such use is meant to reinforce that a fully functional protein is meant to be encompassed by the phrase “polypeptide;” therefore, the use of the phrase “polypeptide or protein” should not be arbitrarily construed as suggesting “polypeptide” and “protein” refer to necessarily distinct embodiments of the invention.

As used herein “isolated” and “substantially isolated” mean, with reference to an invention molecule such as a DNA molecule, amino acid sequence, protein, enzyme, or polypeptide, that said molecule has been altered by the hand of man from its natural state and includes any compound or composition of matter produced with the intention of mimicking, copying or reproducing any compound or composition of matter first identified or characterized from bacteria.

As used herein “DNA molecule” means a nucleic acid or polynucleotide that contains the genetic instructions and encodes for the amino acid sequence of a polypeptide or protein. As used herein, “DNA molecule,” “nucleic acid,” “nucleotide sequence,” and “polynucleotide sequence” are all synonymous.

As used herein, “identity” and “identical” refer to the extent to which two nucleotide or amino acid sequences are invariant.

As used herein, “enzyme” means a polypeptide or protein that is active in catalyzing a chemical or biochemical reaction. Where a protein, or a polypeptide, has or is proposed to have enzymatic activity, the terms “polypeptide,” “protein,” and “enzyme” may be used interchangeably.

As used herein, “maltose binding protein-fatty aldehyde reductase fusion,” abbreviated MBP-FALDR, refers applicants novel fusion protein comprising a maltose binding protein fused to a fatty aldehyde reductase protein, wherein said fatty aldehyde reductase protein comprises an amino acid sequence at least 90%, or at least 95%, or at least 100% identical to the amino acid sequence of SEQ ID NO: 2.

As used herein, “specific activity” is defined as the amount of substrate an enzyme converts (reactions catalyzed), per mg protein in the enzyme preparation, per unit of time.

As used herein, “bacterial origins” means having been first from bacteria and includes any compound or composition of matter produced with the intention of mimicking, copying or reproducing any compound or composition of matter first isolated from bacteria.

As used herein, “fusion protein” means an amino acid sequence (polypeptide or protein) created through the joining of two or more genes or nucleotide sequences which originally coded for separate amino acid sequences.

The following is a summary of exemplar embodiments:

In one embodiment there is provided a DNA molecule isolated from bacteria and comprising a polynucleotide sequence that is at least 90%, or at least 95%, or at least %100 identical to SEQ ID NO: 1. In certain related embodiments said DNA molecule is isolated from the wax ester accumulating bacterium Marinobacter aquaeolei VT8. In certain other related embodiments said DNA molecule encodes for a fatty aldehyde reductase enzyme useful in reducing aldehydes to their corresponding alcohols. In yet other certain related embodiments said DNA molecule encodes for a fatty aldehyde reductase enzyme useful in the production of wax esters. In certain other related embodiments said DNA molecule encodes for an enzyme that comprises a 57 kDa monomer that is capable of reducing a number of long chain aldehydes to corresponding alcohols. In yet other related embodiments said DNA molecule encodes for an enzyme with high specific activity for the reduction of decanal (about 85 nmol decanal reduced/minute/milligram) and may be useful in the production of wax esters.

In another embodiment there is provided a novel fatty aldehyde reductase enzyme isolated from bacteria and comprising an amino acid sequence at least 90%, or at least 95%, or at least 100% identical to SEQ ID NO: 2. In certain related embodiments said fatty aldehyde reductase enzyme is encoded by a polynucleotide sequence at least 90%, at least 95%, or at least 100% identical to SEQ ID NO: 1. In certain other related embodiments the said fatty aldehyde reductase enzyme comprises a 57 kDa monomer that is capable of reducing a number of long chain aldehydes to corresponding alcohols. In yet other related embodiments said fatty aldehyde reductase enzyme shows high specific activity for the reduction of decanal (about 85 nmol decanal reduced/minute/milligram) and may be useful in the production of wax esters. In still other related embodiments said novel fatty aldehyde reductase enzyme is isolated from Marinobacter aquaeolei VT8 and comprises an amino acid sequence at least 90%, or at least 95%, or at least 100% identical to SEQ ID NO: 2 and is encoded by a polynucleotide sequence at least 90%, at least 95%, or at least 100% identical to SEQ ID NO: 1.

In yet another embodiment there is provided an amino acid sequence at least 90%, at least 95%, or at least 100% identical to SEQ ID NO: 2. In certain related embodiments said amino acid sequence is at least 90%, at least 95%, or at least 100% identical to SEQ ID NO: 2 and is encoded by a polynucleotide sequence at least 90%, at least 95%, or at least 100% identical to SEQ ID NO: 1. In certain related embodiments said amino acid sequence at least 90%, at least 95%, or at least 100% identical to SEQ ID NO: 2 provides for a novel fatty aldehyde reductase enzyme isolated from bacteria. In certain related embodiments said amino acid sequence at least 90%, at least 95%, or at least 100% identical to SEQ ID NO: 2 is isolated from the wax ester accumulating bacterium Marinobacter aquaeolei VT8.

In yet another embodiment there is provided a maltose binding protein-fatty aldehyde reductase fusion protein (MBP-FALDR) comprising an amino acid sequence at least 90%, or at least 95%, or at least 100% identical to the amino acid sequence of SEQ ID NO: 3. In certain related embodiments the maltose binding protein-fatty aldehyde reductase fusion protein comprising an amino acid sequence at least 90%, or at least 95%, or at least 100% identical to the amino acid sequence of SEQ ID NO: 3 is encoded by a polynucleotide sequence at least 90%, or at least 95%, or at least 100% identical to the polynucleotide sequence of SEQ ID NO: 4. In related embodiments the amino acid sequence of SEQ ID NO: 3 and the polynucleotide sequence of SEQ ID NO: 4 are of bacterial origin. In certain related embodiments amino acid sequences of the MBP-FALDR fusion protein are at least 90%, or at least 95%, or at least 100% identical to SEQ ID NO: 3 and are encoded by polynucleotide sequences at least 90%, or at least 95%, or at least 100% identical to SEQ ID NO: 4, and are isolated from the wax ester accumulating bacterium Marinobacter aquaeolei VT8.

In yet another embodiment there are provided methods to identify, substantially isolate, characterize and utilize a polynucleotide sequence at least 90%, at least 95%, or at least 100% identical to SEQ ID NO: 1 that further comprises a fatty aldehyde reductase enzyme isolated from the wax ester accumulating bacterium Marinobacter aquaeolei VT8.

In still another embodiment there are provided methods to identify, substantially isolate, characterize and utilize a novel fatty aldehyde reductase enzyme comprising an amino acid sequence at least 90%, at least 95%, or at least 100% identical to SEQ ID NO: 2 and encoded by a nucleotide sequence at least 90%, at least 95%, or at least 100% identical to SEQ ID NO: 1.

The various embodiments of the present invention are useful in, but not necessarily limited to, providing an enzyme of bacterial origin and capable of reducing fatty aldehydes to corresponding fatty alcohols, a useful step in the synthesis of was esters.

### BRIEF DESCRIPTION OF THE DRAWINGS

FIG. 1 shows a four enzyme pathway has been proposed for bacterial wax ester synthesis.

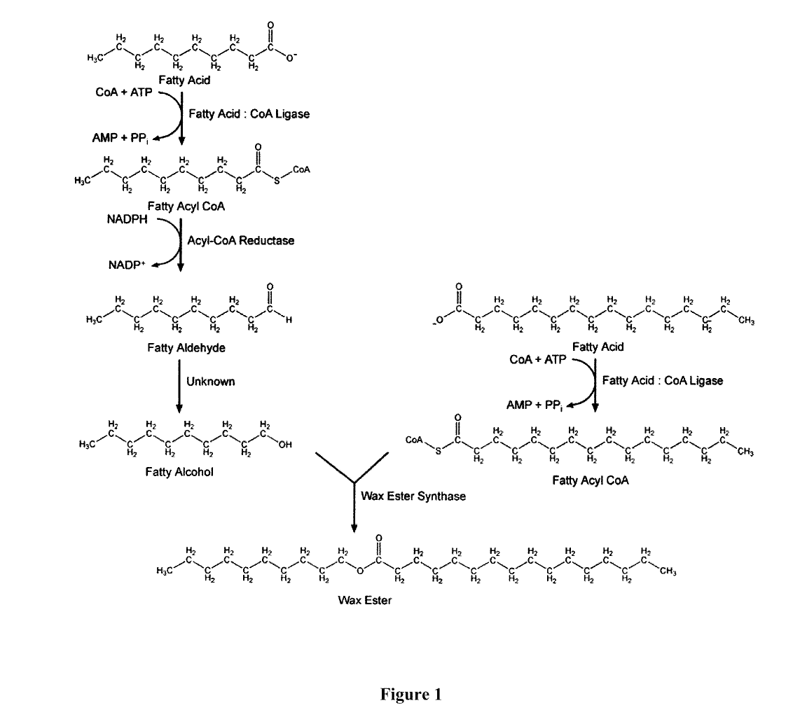

FIG. 2 shows the aldehyde reductase activity of an exemplar embodiment of applicants' maltose binding protein-fatty aldehyde reductase fusion protein (MBP-FALDR) comprising the amino acid sequence of SEQ ID NO: 3 (Km=177 μM, Vₘₐₓ=63 nmol/min/mg).

FIG. 3 is an alignment of the amino acid sequences for the CER4 enzyme from Arabidopsis thaliana and an embodiment of the applicants' novel fatty aldehyde reductase enzyme comprising the amino acid sequence of SEQ ID NO: 2.

### DETAILED DESCRIPTION OF THE INVENTION

The CER4 amino acid sequence from Arabidopsis thaliana was used in a BLAST search of multiple completed bacterial genomes that contain the wax ester synthase (WS/DGAT) gene. Two species of marine bacteria, including Marinobacter aquaeolei VT8 (accession NC_{—}008740.1) were found to contain an open reading frame (ORF) with moderate similarity (48% positive and 27% identical for Marinobacter aquaeolei VT8 to the CER4 gene, however, to the best of applicants' knowledge, no known fatty aldehyde reductase enzyme had previously been isolated and characterized from bacteria; therefore, efforts were made to identify, isolate, to determine the function of and to characterize the polypeptide or protein comprising the amino acid sequence of SEQ ID NO: 2 and encoded by the polynucleotide sequence of SEQ ID NO: 1.

The following materials and methods are useful in practicing the various embodiments of the invention described herein:

All reagents were purchased from Sigma-Aldrich Company (St. Louis, Mo.) unless otherwise specified. Restriction enzymes, T4 DNA ligase and Escherichia coli strain TB1 were obtained from New England Biolabs (Ipswich, Mass.). Bovine serum albumin (BSA) was fraction V (Sigma P/N A2153) and was prepared fresh daily in the same buffer as the assay. NADP⁺-dependant alcohol dehydrogenase from Thermoanaerobium brokii (Sigma P/N A8435) was prepared fresh the day of use for control experiments.

Marinobacter aquaeolei VT8 was obtained from the American Type Cultures Collection (ATCC 700491), and was grown initially on ATCC medium 2084 (Halomonas medium (2)) at 30° C. Plates of the 2084 medium were prepared by adding 1.5% Bacto Agar (B D, Franklin Lakes, N.J.). Genomic DNA was isolated by first growing 1 L of cells to an optical density of 0.6 at 600 nm followed by collection of the cells by centrifugation at 7000 g. The cell pellet was washed once with 50 mM phosphate buffer, pH 7.2. The cells were then suspended in 5 mL of 50 mM Tris-HCl buffer at pH 7.8 with 2% Triton X-100 and 5 mg of lysozyme. The suspension was allowed to sit for 10 minutes at room temperature followed by incubation in boiling water for 5 minutes. The solution was centrifuged at 10,000 g for 10 minutes. The supernatant was retained, and an equal volume of isopropanol was added to precipitate the DNA. The DNA was washed once with 10 mL of isopropanol, and then twice with 10 mL of ice-cold ethanol. The DNA was allowed to air dry and was then subjected to restriction digest and purification following the desalting protocol from the Qiaex II kit (Qiagen, Valencia, Calif.).

A genomic region, proposed by applicants to encode for a fatty aldehyde reductase protein from Marinobacter aquaeolei VT8 (accession NC_{—}008740.1) was amplified from genomic DNA using the primers BBP244 5′ GATGAGGATCCATGGAGCAATACAGCAGGTACATCACGCTGAC and BBP245 5′ GACTGGAATTCAGGCAGCTTTTTTGCGCTGGCGCGC following the Failsafe protocol (Epicenter, Madison, Wis.) using buffer G and an annealing temperature of 60° C. The PCR product was purified using the Qiaex II desalting protocol (Qiagen, Valencia, Calif.) and was digested with EcoRI and NcoI along with the plasmid pBB052 (a pUC19 derivative with kanamycin in place of ampicillin for selection, and an N-terminal His-Tag followed by an NcoI site). The reaction was terminated by heat inactivation at 65° C., followed by ligation. Plasmids were maintained in E. coli strain JM109 unless specified otherwise. The gene encoding for the N-terminal His tagged protein was then transferred to the pET-30A vector to express the proposed fatty aldehyde reductase protein in E. coli strain BL21. Additionally, the EcoRI site following the proposed fatty aldehyde reductase gene in the original vector was removed by digestion with EcoRI, filling in with T4 DNA polymerase, and ligation of the blunt-end product. A new EcoRI site was then introduced just upstream of the second codon of the gene by PCR amplification, removing the methionine start codon, and preparing the gene to be placed in-frame with an EcoRI site following a maltose binding protein from the pMAL-c2x plasmid (New England Biolabs, Ipswich, Mass.). The modified gene was then transferred to the pMAL-c2x plasmid, and sequenced to confirm that it contained no mistakes, prior to transferring this plasmid to the E. coli strain TB1 for expression of the maltose binding protein fused to the proposed fatty aldehyde reductase.

One liter of LB medium supplemented with ampicillin (100 μg/mL) in a 4 L flask was inoculated with 16 mL of an overnight culture of E. coli TB1 transformed with the pMAL-c2x vector expressing the maltose binding protein-fatty aldehyde reductase fusion (MBP-FALDR) protein and was grown for approximately five hours at 37° C. prior to induction by the addition of 50 mg of IPTG (isopropyl β-D-1-thiogalactopyranoside), after which the culture was grown for an additional two hours at room temperature. Cells were harvested by centrifugation and were immediately frozen for later use. The cells were suspended in 30 mL of column buffer (20 mM Tris-HCl pH 7.4, 200 mM NaCl, 1 mM EDTA) supplemented with 0.1 mM phenylmethylsulfonyl fluoride. Cells were lysed by passing the suspended cells through a French pressure cell (SLM Aminco) three times in the presence of DNAse (P/N DN-25, Sigma-Aldrich). Soluble MBP-FALDR protein was collected by centrifuging the cell lysate at 17,500 g for 10 min. The supernatant was diluted three-fold with column buffer and was then applied to a column containing a 10 mL column bed of amylose resin (New England Biolabs, Ipswich, Mass.) and the column was washed with 30 mL of column buffer containing 1 M NaCl, followed by a second wash with 30 mL of column buffer. The MBP-FALDR fusion protein was eluted with 15 mL of column buffer containing 10 mM maltose.

For initial assessment of the activity of the purified MBP-FALDR enzyme, 50 μg of purified MBP-FALDR protein was added to a reaction mixture containing 100 mM of Tris buffer at pH 7.9, 100 mM NaCl, 2.4 mM of either NADPH or NADH as a reductant, and decanal, oleic acid, and hexadecanol as possible substrates. The assays were run under an argon atmosphere in septa sealed vials overnight at room temperature with constant gentle mixing. The products of the reactions were then extracted from the buffer by adding an equal volume of hexane and organic layer components were analyzed by gas chromatography equipped with a flame ionization detector (Forte HT5 column (SGE Analytical Science, Austin, Tex.), 30 meter by 0.32 mm inner diameter with 0.5 μm film thickness, with argon as a carrier and a temperature ramp from 60° C. to 360° C. increasing at 10° C. per minute). Samples containing new peaks not present in a control sample were also run through a gas chromatograph equipped with a mass spectrometer (Shimadzu GC-2010 and GCMS-QP2010S) to assign the identity of the product.

For Fatty Aldehyde Continuous Spectrophotometric Assay Development, all assays were performed in a sealed quartz cuvette with a 1 cm path length. The cuvette and solutions were first degassed with argon on a manifold to remove oxygen from the headspace and solutions. Substrates were initially disbursed into a buffer solution containing BSA at a concentration of 0.5 mg/mL by sonication for three ten second intervals using a microtip sonicator (Branson, Danbury, Conn.). The initial assay for pH optimization included a buffer of 100 mM MOPS, 100 mM MES, and 100 mM TAPS. For reduction assays, 75 μL of a 2 mg/mL NADPH stock was added to bring the initial concentration of NADPH in the cuvette to approximately 200 μM, and an approximate absorbance at 340 nm of 1.2 based on the reported extinction coefficient of 6220 M⁻¹ cm⁻¹ for NADPH. In all assays, the reaction was run for at least two minutes with the NADPH and substrate present, prior to the addition of any enzyme, to obtain a background oxidation rate of the NADPH. The absorbance reading at 340 nm was read every 0.5 seconds, and the reaction was run until a steady-rate was achieved for at least 60 seconds. A linear fit of the data was then used to establish the rate, and the initial background rate was subtracted, to determine the rate associated with substrate reduction by the MBP-FALDR enzyme.

When the assay was used to check for activity with NADH in place of NADPH, NADH was substituted for NADPH at the same concentration as was used for NADPH, and the assay run as described above. Reactions using either NADP⁺ or NAD⁺ also used the same concentration, but it followed the increase in absorbance monitored at 340 nm. Once the pH optimum was established for the enzyme, TAPS and MOPS were removed from the buffer, and only MES was used.

The following is detailed description and discussion of experimental results relating to various embodiments of the present invention and useful in practicing various modes related to the present invention:

### Expression of a Proposed Fatty Aldehyde Reductase:

The gene comprising the nucleotide sequence of SEQ ID NO: 1 and encoding for the putative FALDR enzyme comprising the amino acid sequence of SEQ ID NO: 2 from Marinobacter aquaeolei VT8 was first cloned into a pUC19 derivative vector with an N-terminal 8 His-Tag. Said gene was inserted into a Novagen pET vector (EMD Chemicals, Inc., San Diego, Calif.) to include the N-terminal histidine tag. While initial expression experiments using the pET vector showed a high level of expression of said FALDR enzyme, initial attempts to purify the enzyme were hampered by low solubility. In all cases, the majority of the expressed protein associated with the cell debris following cell disruption and centrifugation. Efforts to improve solubility by inclusion of a variety of common detergents (Tween 20, Triton X-100, Dodecyl Maltoside, and CHAPS) in varied concentrations were met with limited success, and were eventually abandoned for other approaches, as the majority of said FALDR enzyme seemed to remain in the insoluble pellet.

Several approaches were undertaken to improve the solubility of said FALDR enzyme. Eventually, the solubility problem was overcome by creating a fusion protein (MBP-FALDR) utilizing the highly soluble maltose binding protein (MBP). This was accomplished by modifying the gene through PCR to contain an EcoRI restriction site at the beginning of the FALDR nucleotide sequence. This modification removed the methionine residue at the beginning of the amino acid sequence. This modified gene was then inserted into the multiple cloning site of the pMAL-c2x vector (New England Biolabs), which when expressed in the proper E. coli host strain, produced a protein that remained predominantly in the soluble fraction, even without the inclusion of any detergents. This approach allowed a quick purification by using an amylose resin (New England Biolabs, Ipswich, Mass.) to bind the maltose binding protein portion of the fusion protein, and maltose for elution, resulting in a relatively pure protein (approximately 90% pure by SDS-PAGE analysis). The other minor protein components seen in the preparation were presumed to be related to the FALDR, either resulting from proteolytic degradation or premature termination of expression by the host system, as the associated bands were specific to the expression of the MBP-FALDR protein.

### Initial Assessment of the Activity of the Fatty Aldehyde Reductase:

Preliminary experiments were run with the MBP-FALDR enzyme to probe possible substrates by mixing either NADH or NADPH with an aldehyde (decanal), alcohol (hexadecanol) or fatty acid (oleic acid) and then looking for changes in concentration of one or more of the potential substrates by GC analysis. The assays were allowed to run overnight in a sealed vial under an atmosphere of argon to maintain the reduced forms of the nicotinamide coenzymes. Of the possible substrates run in this experiment, only the vial containing NADPH showed a decrease in the decanal peak, and the generation of a new peak, which correlated to retention time for decanol, and was confirmed by mass spectrometry. The reverse reaction using NADP⁺ and hexadecanol or decanol showed no detectable levels of hexadecanal or decanal production. This initial result pointed to the likelihood that the MBP-FALDR enzyme is an NADPH-dependent fatty aldehyde reductase, and further experiments were run to characterize this activity. While none of the experiments performed as part of this work indicated that the MBP-FALDR enzyme is capable of oxidizing the product decanol using NADP⁺, it is possible that this reaction is extremely slow versus the reduction of the aldehyde, and thus is below the level of detection. The activity of medium chain alcohol dehydrogenases from a broad range of species show rates of oxidation of alcohols that are 10% of the rates of the reduction of the corresponding aldehyde, so it is possible that this rate is too low to detect.

### Continuous Assay of Fatty Aldehyde Reductase Activity:

Having established that the MBP-FALDR would utilize NADPH as a substrate, it was possible to employ a continuous spectrophotometric assay to monitor substrate reduction rates based on the loss of absorbance at 340 nm when NADPH is oxidized to NADP⁺. Using this assay, it was possible to establish the pH dependence of the reduction reaction. The rate of reaction was highest at pH 6.3, with a steep increase in activity going from 8.0 to 6.3. The rate at 6.3 was only slightly greater than at 6.5 (30 percent higher). Several factors had to be considered in establishing the optimal pH of the assay. The first was the background oxidation of the NADPH without any MBP-FALDR enzyme present. NADPH naturally degrades to NADP⁺ at neutral and acidic pH values, and is most stable under alkaline conditions. The assays were run under an argon atmosphere to minimize oxidation of the NADPH by O₂. A further consideration was the rapid degradation that occurs at the lowest pH values. While the highest MBP-FALDR enzyme rates were observed at pH 6.3, the rates were highly sensitive to slight pH variations at this value. So, for standard assays used here, a pH of 6.5 was selected, where the activity was minimally affected by slight variations in pH. The following discussion is useful in understanding some features and benefits of the various embodiments of the invention:

### Substrate Specificity and Kinetic Parameters

Six commercially available substrates were examined for reduction by the MBP-FALDR enzyme. The long-chain aldehydes decanal and dodecanal were examined, as well as the smaller aldehydes butanal, hexanal and octanal. The larger, unsaturated aldehyde, cis-11-hexadecenal was also tested. In addition to these straight chain aldehydes, activity was also tested with the aromatic aldehyde benzaldehyde.

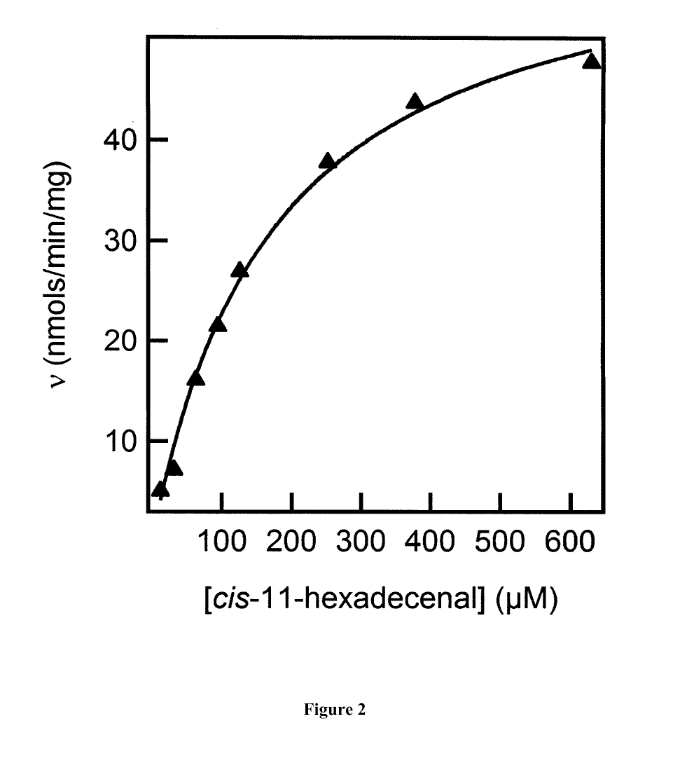

Utilizing the continuous assay to monitor MBP-FALDR activity, the dependence of rate on substrate concentration was determined for all of the commercially available substrates described above. Each assay was repeated multiple times to confirm the results from each single determination. The results obtained for cis-11-hexadecenal are shown in FIG. 2 and are representative of the results obtained for repeated determinations and for the other substrates. The data were fit to the Michaelis-Menten equation, revealing a K_{M} of approximately 177 μM and a maximum velocity of 63 nmol/min/mg. The activity obtained for this substrate was slightly lower than that obtained for decanal. It is difficult to benchmark these rates as no comparable enzyme has been purified. In addition, several factors could limit the observed activity. All of the substrates tested have limited solubility in aqueous solutions, and must first be suspended in solution via sonication. Even under these conditions, it is likely that the substrates exist inside micelles. The sonication process utilized here could result in the partial degradation of the substrate over time. Additionally, the suspension of the substrate in solution is only temporary, and the substrate slowly separates from aqueous solution. Due to these limitations, the measured activity is likely to be an underestimate of the actual activity. In the cell, the enzyme and substrate are likely associated with the hydrophobic membranes. The K_{M} values obtained for each of the substrates that showed activity were in the μM range, indicating that the fatty aldehyde reducatase enzyme comprising the amino acid sequence of SEQ ID NO: 2 should be active in the cell even at low substrate concentrations. It was also observed that the MBP-FALDR enzyme was unstable over time, even when stored at 4° C., losing as much as 50% of the activity over a period of a week. Though the purification is quite rapid, it is uncertain how much activity is lost during preparation.

As can be seen in Table 1, the MBP-FALDR enzyme required a minimal chain length C8 aldehyde (long chain aldehyde) to show significant activity. The shorter substrates butanal (C4) and hexanal (C6) showed no apparent activity under these conditions, while the activity for octanal (C8) was approximately half the activity obtained for decanal (C10), which had the highest activity of the substrates tested (85 nmol/min/mg). A further investigation of the octanal activity revealed that the apparent K_{M} for this substrate may be very close to the concentration tested here (˜750 μM), so that the activity would be greater with higher concentrations of substrate. The activity was lower with dodecanal (C12), though the apparent K_{M} was lower (˜200 μM) than that for octanal and similar to decanal (˜100 μM). In this instance, the solubility may play a more important role, as dodecanal was the longest saturated aldehyde commercially available that was used in this work. The substrate cis-11-hexadecenal (C16) showed activity comparable to decanal, though this substrate does contain a site of unsaturation, which could change accessibility to the active site versus dodecanal. It is also possible that the results here are related more to substrate solubility and availability to the enzyme. As a primary wax ester found in Marinobacter aquaeolei VT8 grown under nitrogen deficient growth conditions is hexadecyl hexadecanoate (unpublished data), it would be expected that hexadecanal would be a likely natural substrate of the fatty aldehyde reducatase enzyme comprising the amino acid sequence of SEQ ID NO: 2. Finally, the aromatic ring containing aldehyde benzaldehyde showed no apparent activity when assayed at similar concentrations, indicating that the active site may be specific for straight chain aldehydes (saturated or unsaturated).

Since all the assays were conducted with the MBP-FALDR fusion protein, a principle concern was whether the fusion protein accurately represented the activity of the wild-type fatty aldehyde reducatase enzyme comprising the amino acid sequence of SEQ ID NO: 2. To examine whether the maltose binding protein affected the activity of the wild-type fatty aldehyde reducatase enzyme comprising the amino acid sequence of SEQ ID NO: 2, the maltose binding protein was removed from the N-terminus of the fatty aldehyde reducatase enzyme comprising the amino acid sequence of SEQ ID NO: 2 by Factor Xa cleavage. The near complete cleavage of maltose binding protein was verified by SDS-PAGE. This cleavage is facilitated by the incorporation of a factor Xa cleavage site (Ile-Glu-Gly-Arg) just upstream of the EcoRI site of the pMAL-c2x vector. Assays conducted with decanal and the cleaved fatty aldehyde reducatase enzyme comprising the amino acid sequence of SEQ ID NO: 2, no longer fused to MBP, did not reveal any loss or improvement in the rate of substrate reduction from the rate exhibited by the MBP-FALDR fusion protein; therefore, it is expected the results shown for the MBP-FALDR protein are representative of the in-vivo activity of the fatty aldehyde reducatase enzyme comprising the amino acid sequence of SEQ ID NO: 2

Reversibility of the Enzyme with Fatty Alcohols

To test the possible reversibility of the fatty aldehyde reducatase enzyme comprising the amino acid sequence of SEQ ID NO: 2, the oxidation of a number of alcohols to the corresponding aldehyde using NADP⁺ as the oxidant were tested. These tests were performed with the MBP-FALDR enzyme. In these assays, the increase in absorbance at 340 nm was followed. Under no conditions did any reduction of NADP⁺ occur in the presence of the fatty alcohols tested. As a control, the alcohol dehydrogenase from Thermoanaerobium brokii was followed with 2-propanol and the same stock of NADP⁺ to confirm the integrity of the assay. These results showed no evidence that MBP-FALDR is capable of catalyzing the reverse reaction, the oxidation of a fatty alcohol to a fatty aldehyde. Similarly, reactions were followed in the same manner by substituting NADH or NAD⁺ for NADPH or NADP⁺ using decanal and decanol, respectively, with no indication of activity over background with any combination except with decanal and NADPH. Based on this, it is proposed that the fatty aldehyde reducatase enzyme comprising the amino acid sequence of SEQ ID NO: 2 exhibits activity only for the reduction of fatty aldehydes in an NADPH-dependent reaction.

### Inhibition Studies

To probe the possible mechanism of the fatty aldehyde reducatase enzyme comprising the amino acid sequence of SEQ ID NO: 2, applicants again used the MBP-FALDR enzyme and some potential chemical inhibitors were tested for effects on the reduction of decanal by the MBP-FALDR enzyme in the presence of NADPH. In all cases, the compound was tested initially at concentrations of 1.0 mM, and if a significant inhibition was found, lower concentrations were also tested (Table 2). The metal chelator EDTA, which is used during purification of the MBP-FALDR enzyme to limit the activity of metalloproteases, had little effect on the activity. Reductants such as ascorbic acid, dithiothreitol and β-mercaptoethanol, also showed little effect (less than 25% decrease in activity). Only the use of dithionite resulted in a significant decrease in activity of the MBP-FALDR enzyme. At higher concentrations, this was difficult to assess fully, as the dithionite interferes with the absorbance at 340 nm where activity is measured. At the lower concentration of 250 μM, the interference is lower, and the inhibition is more pronounced. This could be an indication of an active site residue or cofactor that is susceptible to reduction. The two metal chelators dipyridyl and diethyldithiocarbamate showed only a moderate inhibition of activity at elevated concentrations of 1.0 mM. This would indicate that if a transition metal is involved in the catalysis, it is not readily accessible to such chelators. Finally, the ability of decanol to inhibit reduction of decanal was also tested. Here, inhibition of almost 45% at the two concentrations tested was observed, indicating a possibility that product inhibition can regulate activity, even though the MBP-FALDR enzyme is apparently not reversible.

Applicants have demonstrated that Marinobacter aquaeolei VT8 contains a gene that encodes a fatty aldehyde reductase enzyme comprising the amino acid sequence of SEQ ID NO: 2. To applicants' knowledge, this is the first report of the isolation and characterization of a bacterial enzyme that is capable of reducing fatty aldehydes to corresponding alcohols.

### Detailed Embodiments of the Invention

In one embodiment there are provided polynucleotide sequences at least 90%, at least 95%, or at least 100% identical to the polynucleotide sequence of SEQ ID NO: 1 and that encode for certain amino acid sequences at least 90%, or at least 95%, or at least 100% identical to the amino acid sequence of SEQ ID NO: 2, wherein said amino acid sequence of SEQ ID NO: 2 provides for a polypeptide or protein comprising a fatty aldehyde reductase enzyme. In one embodiment, said fatty aldehyde reductase enzyme is a 57 kDa monomer that is capable of reducing a number of long chain aldehydes to corresponding alcohols. In a related embodiment the polynucleotide sequences at least 90%, at least 95%, or at least 100% identical to the polynucleotide sequence of SEQ ID NO: 1 are isolated from the wax ester accumulating bacterium Marinobacter aquaeolei VT8.

In yet another embodiment there is provided a maltose binding protein-fatty aldehyde reductase fusion protein (MBP-FALDR) comprising an amino acid sequence at least 90%, or at least 95%, or at least 100% identical to the amino acid sequence of SEQ ID NO: 3. In certain related embodiments the maltose binding protein-fatty aldehyde reductase fusion protein comprising an amino acid sequence at least 90%, or at least 95%, or at least 100% identical to the amino acid sequence of SEQ ID NO: 3 is encoded by a polynucleotide sequence at least 90%, or at least 95%, or at least 100% identical to the polynucleotide sequence of SEQ ID NO: 4. In related embodiments the amino acid sequence of SEQ ID NO: 3 and the polynucleotide sequence of SEQ ID NO: 4 are of bacterial origin. In certain related embodiments amino acid sequences of the MBP-FALDR fusion protein are at least 90%, or at least 95%, or at least 100% identical to SEQ ID NO: 3 and are encoded by polynucleotide sequences at least 90%, or at least 95%, or at least 100% identical to SEQ ID NO: 4, and are isolated from the wax ester accumulating bacterium Marinobacter aquaeolei VT8.

The relative activity levels of an exemplar fatty aldehyde reductase enzyme, the maltose binding protein-fatty aldehyde reductase fusion protein comprising the amino acid sequence of SEQ ID NO: 3, for specific fatty aldehyde substrates are shown in Table 1.

Referring now to Table 1, all activity assays were measured at a substrate concentration of 100 μg/mL following the assay protocol described in material and methods disclosed in this application and using 160 μg of fatty aldehyde reductase enzyme. The activity measurement for each substrate was performed multiple times to confirm reproducibility. Activities reported here are the results from a single set of data. The activity for decanal was 85 nmols/min/mg, and all other activities were divided by this result and multiplied by 100.

TABLE 1
Fatty Aldehyde Reductase Substrate Comparisons

| | | Relative |
|---|---|---|
| | | Activity |
| | | (Percent of |
| Substrate* | Substrate Molecular Structure | Decanal) |
| Butanal | 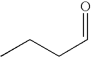 | <1 |
| Hexanal | 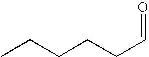 | <1 |
| Octanal | 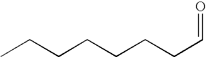 | 52 |
| Decanal | 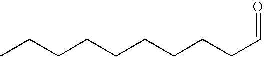 | 100 |
| Dodecanal | 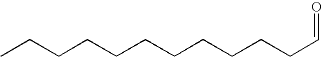 | 55 |
| cis-11-hexadecenal | 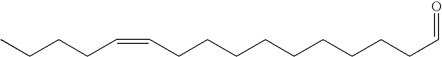 | 87 |
| Benzaldehyde | 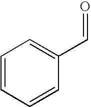 | <1 |

Certain embodiments of the invention may be characterized, in part, by the inhibition of the reduction of decanal by a fatty aldehyde reductase enzyme comprised by an amino acid sequence at least 90% identical to the amino acid sequence of SEQ ID NO: 2. As described previously, it may be desirable to Table 2 shows the inhibition of decanal reduction for the MBP-FALDR comprising the amino acid sequence of SEQ ID NO: 3 and further comprising a polypeptide or protein that is a 57 kDa monomer and that exhibits the relative activity for the reduction of decanal as shown in Table 1.

TABLE 2
Inhibition of Decanal Reduction

Inhibitor Concentration

| | 1.0 mM | 0.25 mM |
|---|---|---|
| | (Percent Activity) | (Percent Activity) |
| EDTA (Disodium Salt) | 81 | ND* |
| Ascorbic Acid | 76 | ND* |
| Dithiothreitol | 76 | ND* |
| β-Mercaptoethanol | 75 | ND* |
| Dithionite | 25 | 8 |
| Diethyldithiocarbamate | 50 | 61 |
| Dipyridyl | 46 | 59 |
| Dodecanol | 54 | 55 |

Referring again to Table 2, the inhibition assays were run in 100 mM MES buffer pH 6.5 with 200 μM NADPH, 250 μM decanal and 125 μg of MBP-FALDR fusion protein. All compounds were dissolved in buffer except dipyridyl, which was dissolved in dimethyl sulfoxide (DMSO). Inclusion of DMSO did not result in decreased activity at the same concentration. Activity is reported as the activity remaining in the presence of each compound as a percentage of the activity without any inhibitor present. *Not determined. Each compound was tested multiple times to confirm reproducibility. The remaining activity presented here is the result from a single set of data.

In another embodiment there is provided a DNA molecule comprising a polynucleotide sequence at least 90%, or at least 95%, or at least 100% identical to SEQ ID NO: 1 and that encodes for a fatty aldehyde reductase enzyme. In certain related embodiments said DNA molecule is isolated from the wax ester accumulating bacterium Marinobacter aquaeolei VT8. In certain other related embodiments said DNA molecule encodes for a fatty aldehyde reductase enzyme that is a 57 useful in reducing aldehydes to corresponding alcohols. In yet other certain embodiments said DNA molecule encodes for a fatty aldehyde reductase useful in the production of wax esters.

In another embodiment there is provided a novel fatty aldehyde reductase enzyme of bacterial origin and comprising the amino acid sequence at least 90%, at least 95%, or at least 100% identical to SEQ ID NO: 2. In certain related embodiments the fatty aldehyde reductase enzyme of bacterial origin and comprising the amino acid sequence at least 90%, at least 95%, or at least 100% identical to SEQ ID NO: 2 further comprises the primary, secondary, tertiary, or quaternary protein structure of a fatty aldehyde reductase enzyme. In other related embodiments said fatty aldehyde reductase enzyme is encoded for by a nucleotide sequence at least 90%, at least 95%, or at least 100% identical to SEQ ID NO: 1. In certain other embodiments the said fatty aldehyde reductase enzyme comprises a 57 kDa monomer that is capable of reducing a number of long chain aldehydes to corresponding alcohols. In yet other certain embodiments said fatty aldehyde reductase enzyme may show high specific activity for the reduction of decanol (85 nmol decanal reduced/minute/milligram) and may be useful in the production of wax esters.

In certain related embodiments there is provided an amino acid sequence at least 90%, at least 95%, or at least 100% identical to SEQ ID NO: 2 and further comprising the secondary, tertiary or quaternary structure of a polypeptide or protein further comprising an enzyme with fatty aldehyde reductase activity. Primary protein structure is generally defined as the linear sequence of amino acids comprising a polypeptide chain. The amino acid sequence is determined by the sequence of nucleotide bases in the DNA that encode the polypeptide chain. The bond between two amino acids is a peptide bond and the sequence of amino acids determines the positioning of the different amino acid R groups relative to each other. The positioning of the R groups determines the way that the protein folds and the final structure of the protein or enzyme. The secondary structure of a polypeptide or protein is determined by hydrogen bonds forming between the atoms of the amino acid backbone of the polypeptide chain, which results in characteristic twisting of the polypeptide chain. Alpha helix or B pleated sheets are common secondary structures. The tertiary structure of a polypeptide or protein is the three dimensional globular structure formed by bending, twisting and folding of the polypeptide chain, which can result in the linear sequence of amino acids being folded into a compact globular structure. Commonly, the folding of the polypeptide chain is stabilized by multiple weak, noncovalent interactions. These interactions include hydrogen bonds, electrostatic interactions, hydrophobic interactions and covalent bonds. Commonly, the covalent bonds include disulfide bonds between two cysteines adjacent to each other during the bending, twisting and folding of the polypeptide chain. Commonly, the quaternary structure of a protein refers to the overall structure of a protein that contains more than one polypeptide chain. As used herein, when referring to a polypeptide chain comprising part or all of a protein, quaternary structure refers the amino acid sequence of said polypeptide chain in the context of the quaternary structure of said protein. Said differently, quaternary structure, again referring to a polypeptide chain comprising part or all of a protein, refers to polypeptide of the present invention in association with other polypeptide chains of similar or different amino acids sequence.

In yet another embodiment there are provided methods to identify, substantially isolate or purify, characterize and utilize a polynucleotide sequence at least 90%, at least 95%, or at least 100% identical to SEQ ID NO: 1 that encodes a fatty aldehyde reductase enzyme isolated from the wax ester accumulating bacterium Marinobacter aquaeolei VT8.

In still another embodiment there are provided methods to identify, substantially isolate or purify, characterize and utilize a novel fatty aldehyde reductase enzyme comprising an amino acid sequence at least 90%, at least 95%, or at least 100% identical to SEQ ID NO: 2 and encoded by a nucleotide sequence at least 90%, at least 95%, or at least %100 identical to SEQ ID NO: 1.

In one embodiment there is provided a fatty aldeyhyde reductase protein with enzymatic activity for the reduction of a fatty aldehyde to a corresponding fatty alcohol. No bacterial gene encoding this class of enzyme has been previously described in the scientific literature.

Various embodiments of the invention relate to the cloning and expression of a polynucleotide gene sequence at least 90%, at least 95%, or at least 100% identical to the nucleotide sequence of SEQ ID NO: 1 and further comprising a polynucleotide gene sequence from the marine bacterium Marinobacter aquaeolei VT8 that encodes a polypeptide further comprising a protein shown to have fatty aldehyde reductase activity. In some embodiments said polynucleotide gene sequence is useful in providing a polypeptide or protein active in the proposed pathway for wax ester synthesis (see Scheme 1). The gene for this FALDR was identified by searching bacterial genomes with the gene CER4 sequence from the plant Arabidopsis. Marinobacter species are known to accumulate branched wax esters when grown in a media supplemented with phytol, but detailed reports regarding production of wax esters from simple sugars have not been reported. The product of the fatty aldehyde reductase gene in Marinobacter aquaeolei VT8 shares some degree of primary sequence identity with the CER4 gene from Arabidopsis. A fatty aldehyde reductase gene comprising the polynucleotide sequence of SEQ ID NO: 1 has been cloned from Marinobacter aquaeolei VT8 and the polypeptide encoded by said polynucleotide sequence and further comprising the amino acid sequence of SEQ ID NO: 2 has been purified as a fusion protein with a maltose binding protein. A description of a fatty aldehyde reductase enzyme comprising the polypeptide encoded by the polynucleotide sequence of SEQ ID NO: 1 and further comprising the amino acid sequence of SEQ ID NO: 2, is provided herein. A discussion of the substrate specificity of said fatty aldehyde reductase enzyme is also provided herein.

In some embodiments the present invention relates to a fatty aldehyde reductase enzyme active in the synthesis of wax esters. The synthesis of wax esters from fatty acids is proposed to require the action of a four enzyme pathway. An essential step in the pathway is the reduction of a fatty aldehyde to a corresponding fatty alcohol, although the enzyme responsible for catalyzing this reaction has never before been identified in bacteria. Provided herein are methods for the purification and characterization of an enzyme from the wax ester accumulating bacterium Marinobacter aquaeolei VT8, which functions as a fatty aldehyde reductase in this pathway.

In further embodiments of the invention there is provided a polynucleotide gene sequence and an amino acid sequence relating to a polypeptide molecule exhibiting fatty aldehyde reductase.

In still further embodiments there is provided an enzyme that is an NADPH-dependent fatty aldehyde reductase that is capable of activity with a variety of substrates.

In yet still further embodiments there is provided a highly soluble fatty aldehyde reductase protein formed by the fusion of FALDR to Maltose Binding Protein (MBP).

Referring now to the invention in still more detail, in FIG. 2 there is shown substrate saturation for the maltose binding protein-fatty aldehyde reductase (MBP-FALDR) fusion protein. Aldehyde reductase activity of MBP-FALDR was measured spectrophotometrically by monitoring the oxidation of NADPH continuously at 340 nm. Absorbances versus time measurements were used to determine initial rates of aldehyde reduction at each concentration of cis-11-hexadecenal. The initial rates were fit to the Michaelis-Menten equation to determine Vₘₐₓ and K_{M}. K_{M} was determined to be 177 μM, and Vₘₐₓ was determined to be 63 nmol/min/mg. Experiments were repeated three times to confirm reproducibility. The results shown here represents the data obtained from a single set of data.

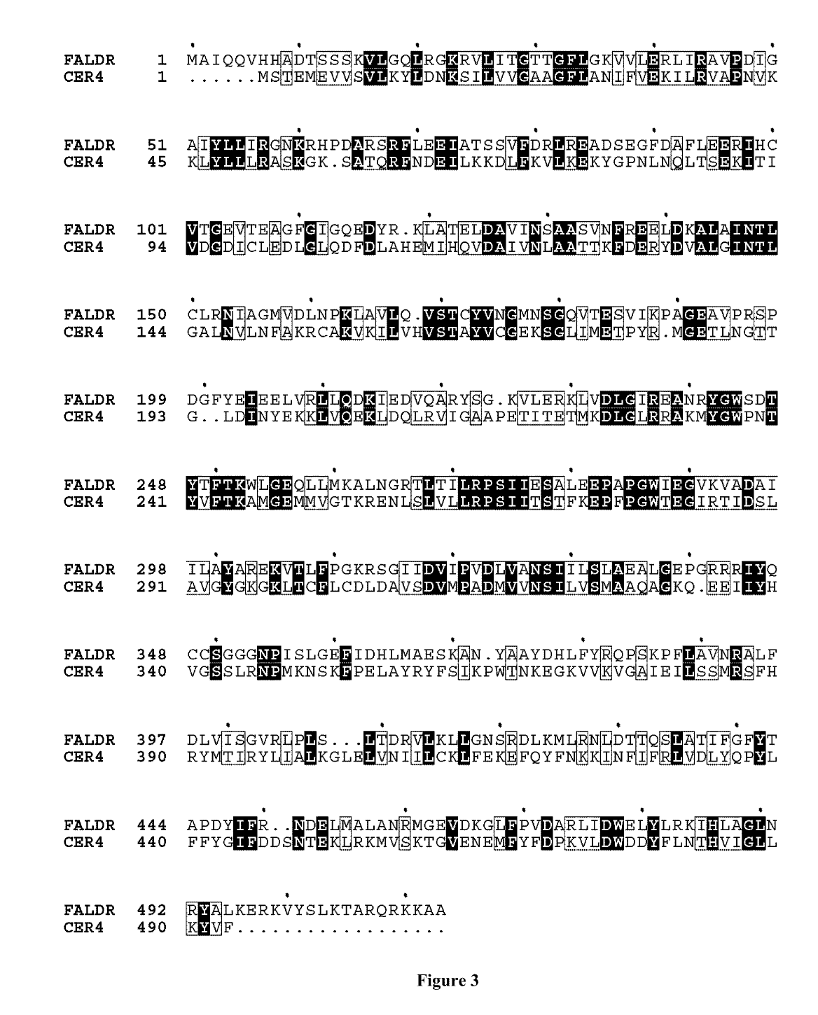

Referring now to the invention in still more detail, in FIG. 3 there is shown an alignment of the Marinobacter aquaeolei fatty aldehyde reductase protein comprising SEQ ID NO: 2 and the CER4 protein. There is moderate similarity between the two proteins. The black squares residues that are identical and the boxed residues identify residues that are chemically similar, such as similar polarities, charges, aromaticity or hydrophobicity
Referring now to an SDS-PAGE for a fatty aldehyde reductase related to the present invention. SDS-PAGE analysis of E. coli BL21 cells expressing an N-terminal histidine tagged FALDR was performed. A band with an approximate molecular weight of 55 kDa comprises 80% of the insoluble fraction of the cell free lysate. No corresponding band appears in the soluble fraction of the cell free lysate indicating that all of the overexpressed FALDR protein is found in the insoluble fraction. SDS-PAGE analysis of the purification of MBP-FALDR from E. coli TB1 cells demonstrates the importance of the fusion protein to facilitate purification. The insoluble fraction of the cell free lysate does not contain a band that would be consistent with a protein the size of the MBP-FALDR fusion protein. The soluble fraction does however contain a band which has an approximate molecular weight greater than 83 kDa and appears to be overexpressed. The final purification step contains two minor bands with approximate molecular weights of 40 and 45 kDa and a prominent band with an approximate molecular weight greater than 83 kDa.

### EXAMPLES

Discussed below are various examples of the various embodiments of the present invention. The various embodiments may be useful alone or in combination with each other or as a whole. Prior to the discussion of specific examples there is provided a disclosure pertaining to various broad embodiments of the various embodiments of the present invention.

Disclosed herein are polynucleotide and amino acid polypeptide sequences useful in the reduction of aldehydes into corresponding alcohols. Said reduction of aldehydes into corresponding alcohols is a key step in the biosynthesis of wax esters and said nucleotides and polypeptides are therefore useful in the synthesis of wax esters by enzymatic, biochemical or biosynthetic means.

Those skilled in the art will appreciate that due to the redundancy in the genetic code a polynucleotide sequence can be substantially altered without changing the corresponding amino acid polypeptide encoded by said nucleotide sequence; therefore, a second nucleotide sequence, being derived from a first nucleotide sequence without substantially changing the amino acid polypeptide encoded by the first nucleotide sequence, can at once be envisioned by those skilled in the art.

Those skilled in the art will also appreciate that homologues or derivatives of the polypeptides or proteins disclosed in the various embodiments of the present invention may be modified by the known methods in the art which include the substitution, deletion or addition of amino acids. Modifications may also include the fusion of the polypeptides or proteins related to the present invention to a second polypeptide or protein where such fusion alters the properties of the polypeptides or proteins of the present invention without eliminating the functionality of the polypeptides or proteins related to the present invention. Without limiting the invention in any way, an example of such fusion of the polypeptides or proteins related to the present invention to a second polypeptide or protein is embodied in the fusion of Maltose Binding Protein to the polypeptides or proteins related to the present invention.

For amino acid identity or similarity for an optimal alignment, a program like BLASTx will align the longest stretch of similar sequences and assign a value to the fit. It is thus possible to obtain a comparison where several regions of similarity are found, each having a different score. Both types of analysis are contemplated in the present invention.

Homologues or derivatives having at least 70%, at least 80%, at least 90% or even 95% identity can be envisioned by one in the art, however, for homologues and derivatives of the polypeptides or proteins of the present invention, the degree of identity or similarity with a protein or polypeptide as described above is less important than that the homologue or derivative should retain the fatty aldehyde reductase activity of the polypeptides or proteins related to the present invention.

The nucleic acid molecules related to the various embodiments of the present invention include novel variants of the nucleic acid molecules particularly disclosed herein. One skilled in the art would recognize such variants are encompassed by the present invention. Said variants may occur in nature as a result of natural variation. For example, additions, substitutions and/or deletions of one or more nucleotides could be envisioned by one skilled in the art. Said variants may also arise from the work of one skilled in the art. Thus, synthetic or non-naturally occurring variants are also included within the scope of the invention.

Biotechnological techniques common to the art are also useful in the isolation or utilization of the various embodiments of the invention disclosed herein. Without limiting the invention in any way, one example is expression cloning, which is discussed herein. In expression cloning, a nucleotide sequence coding for a protein of interest is cloned into an expression vector. Commonly the cloning of the protein is by the use of using PCR and restriction enzymes. The expression vector into which the nucleotide sequence is cloned is commonly, but not necessarily, a plasmid. Commonly the expression vector comprises the nucleotide sequence encoding the protein of interest and special promoter elements to drive production of the protein of interest, and may also have antibiotic resistance markers.

An expression vector comprising the nucleotide sequence encoding the protein of interest may be inserted into either bacterial or eukaryotic cells. Without limiting the present invention, nucleotide sequences related to the present invention may be introduced into bacterial cells can by transformation, conjugation or by transduction. Again without limiting the invention, nucleotide sequences related to the present invention may be introduced into eukaryotic cells by physical or chemical means commonly known as transfection. In one embodiment transfection is by calcium phosphate transfection, whereas in another embodiment it is by electroporation, and in a third embodiment by microinjection, and in yet another embodiment by liposome transfection. In still further embodiments of the present invention, nucleotide sequences of the present invention may also be introduced into eukaryotic cells using viruses or bacteria as carriers in a process commonly termed bactofection.

### Example 1

In one exemplar embodiment there is provided an isolated DNA molecule from Marinobacter aquaeolei VT8 and comprising the polynucleotide sequence of SEQ ID NO: 1 and encoding a polypeptide comprising the amino acid sequence of SEQ ID NO: 2. The DNA molecule of SEQ ID NO:1 may be provided for as a single-stranded DNA. Alternatively, the DNA molecule of SEQ ID NO: 1 may be provided in a double-stranded DNA, wherein the DNA molecule of SEQ ID NO: 1 is bound to a complementary strand of DNA.

### Example 2

In another exemplar embodiment there is a DNA molecule comprising a polynucleotide sequence at least 90%, at least 95%, or at least 100% identical to the polynucleotide sequence of SEQ ID NO: 1. Those skilled in the art would recognize that due to natural variations in polynucleotide gene sequences and the presence of various alleles for such polynucleotide gene sequences, even a single species of bacterium may have slight differences in the polynucleotide sequence for any given gene. Due to redundancy in the genetic code, slight changes in the polynucleotide sequence for any given gene might or might not result in a change to the amino acid sequence of the polypeptide or protein encode by said polynucleotide sequence. Even when the amino acid sequence encoded by the polynucleotide is changed, those skilled in the art would recognize many, if not all, of the changes that would likely cause little or no change in the function of the polypeptide or protein provided for by the amino acid sequence. Furthermore, because of the redundancy of the genetic code and the existence of well known techniques for manipulating a known polynucleotide sequence, and given the disclosure of the polynucleotide sequence of SEQ ID NO: 1, those skilled in the art would be able to at once envision and obtain, without undue experimentation, certain various embodiments of the present invention that would retain the fatty aldehyde reductase activity of the polypeptide encoded by the polynucleotide sequence of SEQ ID NO: 1.

### Example 3

In yet another embodiment there is provided the amino acid sequence of SEQ ID NO: 2, which further comprises a polypeptide that provides for an enzyme with aldehyde reductase activity. This embodiment may include related bacterial polypeptide sequences of enzymes with aldehyde reductase activity.

### Example 4

Another embodiment of the invention is modified forms of the polynucleotide of SEQ ID NO: 1 and polypeptide sequence of SEQ ID NO: 2 that encode for or comprise the primary structure of an enzyme with aldehyde reductase activity. Modifications may include those modifications deemed necessary for routine engineering and handling of the nucleotide sequence, polypeptide sequence or enzyme embodiments of the invention. Modifications may also include those modification routinely made to improve the performance of the nucleotide sequence, polypeptide sequence or enzyme embodiment of the invention. One specific modification disclosed herein is the FALDR-MBP fusion protein comprising the amino acid sequence of SEQ ID NO: 3 and encoded by the polynucleotide sequence of SEQ ID NO: 4.

### Example 5

Another embodiment of the invention is a fatty aldehyde reductase enzyme comprising a 57 kDa monomer that is capable of reducing a number of long chain aldehydes to the corresponding alcohols. In yet other certain embodiments said fatty aldehyde reductase enzyme may show high specific activity for the reduction of decanal (85 nmol decanal reduced/minute/milligram) and may be useful in the production of wax esters.

The various embodiments and examples described in this application may be useful alone or in any combination with each other.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

We claim:

1. An isolated DNA molecule, comprising: a nucleic acid molecule isolated from bacteria and encoding a polypeptide or protein having fatty aldehyde reductase activity and comprising a polynucleotide sequence at least 90% similar to SEQ ID NO: 1.

2. The isolated DNA molecule of claim 1, further comprising: an isolated nucleic acid molecule encoding a polypeptide or protein having fatty aldehyde reductase activity and comprising a nucleotide sequence at least 95% similar to SEQ ID NO: 1.

3. The isolated DNA molecule of claim 1, further comprising: an isolated nucleic acid molecule encoding a polypeptide having fatty aldehyde reductase activity and comprising the nucleotide sequence of SEQ ID NO: 1.

4. The isolated DNA molecule of claim 1, further comprising: an isolated nucleic acid molecule encoding for an amino acid sequence at least 90%, or at least 95%, or at least 100% identical to SEQ ID NO: 2, wherein said isolated nucleic acid molecule is isolated from Marinobacter aquaeolei VT8, and wherein said isolated nucleic acid molecule further comprises a polynucleotide that encodes for an enzyme comprising a protein that is a 57 kDa monomer, wherein said enzyme has specific activity for the reduction of decanal of about 85 nmol decanal reduced/minute/milligram.

5. The isolated DNA molecule of claim 2, further comprising: an isolated nucleic acid molecule encoding for an amino acid sequence at least 90%, or at least 95%, or at least 100% identical to SEQ ID NO: 2, wherein said isolated nucleic acid molecule is isolated from Marinobacter aquaeolei VT8, and wherein said isolated nucleic acid molecule further comprises a polynucleotide that encodes for an enzyme comprising a protein that is a 57 kDa monomer, wherein said enzyme has specific activity for the reduction of decanal of about 85 nmol decanal reduced/minute/milligram.

6. The isolated DNA molecule of claim 3, further comprising: an isolated nucleic acid molecule encoding for an amino acid sequence at least 90%, or at least 95%, or at least 100% identical to SEQ ID NO: 2, wherein said isolated nucleic acid molecule is isolated from Marinobacter aquaeolei VT8, and wherein said isolated nucleic acid molecule further comprises a polynucleotide that encodes for an enzyme comprising a protein that is a 57 kDa monomer, wherein said enzyme has specific activity for the reduction of decanal of about 85 nmol decanal reduced/minute/milligram.

7. An isolated fatty aldehyde reductase enzyme, comprising: a polypeptide or protein of bacterial origins, wherein said polypeptide or protein comprises an amino acid sequence at least 90% identical to the amino acid sequence of SEQ ID NO: 2.

8. The isolated fatty aldehyde reductase enzyme of claim 7, further comprising: a polypeptide or protein of bacterial origins, wherein said polypeptide or protein comprises an amino acid sequence at least 95% identical to the amino acid sequence of SEQ ID NO: 2.

9. The isolated fatty aldehyde reductase enzyme of claim 7, further comprising: a polypeptide or protein of bacterial origins, wherein said polypeptide or protein comprises the amino acid sequence of SEQ ID NO: 2.

10. The isolated fatty aldehyde reductase enzyme of claim 7, further comprising: an enzyme that is a 57 kDa monomer, wherein said enzyme has a specific activity for the reduction of decanal of about 85 nmol decanal reduced/minute/milligram and wherein said enzyme is encoded by a nucleotide sequence at least 90%, or at least 95%, or at least 100% identical to SEQ ID NO: 1.

11. The isolated fatty aldehyde reductase enzyme of claim 8, further comprising: an enzyme that is a 57 kDa monomer, wherein said enzyme has a specific activity for the reduction of decanal of about 85 nmol decanal reduced/minute/milligram and wherein said enzyme is encoded by a nucleotide sequence at least 90%, or at least 95%, or at least 100% identical to SEQ ID NO: 1.

12. The isolated fatty aldehyde reductase enzyme of claim 9, further comprising: an enzyme that is a 57 kDa monomer, wherein said enzyme has a specific activity for the reduction of decanal of about 85 nmol decanal reduced/minute/milligram and wherein said enzyme is encoded by a nucleotide sequence at least 90%, or at least 95%, or at least 100% identical to SEQ ID NO: 1.

13. The isolated fatty aldehyde reductase enzyme of claim 7, 8, 9 or 10, further comprising: a fatty aldehyde reductase enzyme of claim 7, 8, 9 or 10 fused to a maltose binding protein.

14. A fusion protein, comprising: a maltose binding protein-fatty aldehyde reductase fusion protein comprising an amino acid sequence at least 90% identical to the amino acid sequence of SEQ ID NO: 3, wherein the amino acid sequence of SEQ ID NO: 3 is encoded by a polynucleotide sequence at least 90% identical to the polynucleotide sequence of SEQ ID NO: 4.

15. The fusion protein of claim 14, further comprising: a maltose binding protein-fatty aldehyde reductase fusion protein comprising an amino acid sequence at least 95% identical to the amino acid sequence of SEQ ID NO: 3, wherein the amino acid sequence of SEQ ID NO: 3 is encoded by a polynucleotide sequence at least 90% identical to the polynucleotide sequence of SEQ ID NO: 4.

16. The fusion protein of claim 14, further comprising: a maltose binding protein-fatty aldehyde reductase fusion protein comprising an amino acid sequence at least 95% identical to the amino acid sequence of SEQ ID NO: 3, wherein the amino acid sequence of SEQ ID NO: 3 is encoded by a polynucleotide sequence at least 95% identical to the polynucleotide sequence of SEQ ID NO: 4.

17. The fusion protein of claim 14, further comprising: a maltose binding protein-fatty aldehyde reductase fusion protein comprising the amino acid sequence of SEQ ID NO: 3, wherein the amino acid sequence of SEQ ID NO: 3 is encoded by the polynucleotide sequence of SEQ ID NO: 4.


SEQUENCE LISTING (4 sequences)

SEQ ID NO: 1 - DNA, 1542 bp, Marinobacter aquaeolei VT8
atggcaatacagcaggtacatcacgctgacacttcatcatcaaaggtgctcggacagctccgtggcaagcgggttctgatcaccggtaccactggctttctgggcaaggtggtcctcgaaaggctgattcgggcggtgcctgatatcggcgcaatttacctgctgatccggggcaataaacggcatccggatgctcgttcccgtttcctggaagaaattgccacctcctcggtgtttgaccgtcttcgcgaggccgattcagagggatttgacgcctttctggaagagcgcattcactgcgtgaccggtgaggtgaccgaagcgggtttcgggatagggcaggaagactatcgcaaactcgccaccgaactggatgcggtgatcaactccgctgcaagcgtgaatttccgtgaagagctcgacaaggcgctggccatcaacaccctgtgccttcggaatattgccggcatggtggatttgaatccgaagcttgcggtcctgcaggtctccacctgctatgtcaatggcatgaactcggggcaggtaaccgaatcggtgatcaagccggcaggcgaggccgtgccgcgttccccggacggcttctatgagatagaagagcttgttcgcctgcttcaggataaaattgaagacgttcaggcccgttattccggcaaagtgctggagaggaagctggtggacctggggattcgggaagccaaccgctatggctggagcgatacctacacctttaccaagtggctgggcgaacagttgctgatgaaggcgttaaacgggcgcacgctgaccattctgcgtccttcgattatcgaaagtgccctggaggaaccagcgcccggctggattgagggggtgaaggtggcagatgccatcatcctggcttacgcacgggaaaaagtcaccctcttcccgggcaaacgctccggtatcatcgatgtgattccagtggacctggtggccaactccatcatcctttccctggcggaagctcttggagaacccggtcgacgtcgcatctatcaatgttgcagcgggggcggcaatccaatctccctgggtgagttcatcgatcatctcatggcggaatcaaaagccaattacgctgcctacgatcacctgttctaccggcagcccagcaagccgtttctggcggttaaccgggcgctgtttgatttggtgatcagtggtgttcgcttaccgctctccctgacggaccgtgtgctcaaattactgggaaattcccgggacctgaaaatgctcaggaatctggataccacccagtcgctggcaaccatttttggtttctacaccgcgccggattatatcttccggaacgatgagctgatggcgctggcgaaccggatgggtgaggtcgataaagggctgttcccggtggatgcccgcctgattgactgggagctctacctgcgcaagattcacctggccgggctcaatcgctatgccctgaaagaacgaaaggtgtacagtctgaaaaccgcgcgccagcgcaaaaaagctgcctga

SEQ ID NO: 2 - protein, 513 aa, Marinobacter aquaeolei VT8
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 3 - protein, 903 aa, Marinobacter aquaeolei VT8
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 4 - DNA, 2712 bp, Marinobacter aquaeolei VT8
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgcaatacagcaggtacatcacgctgacacttcatcatcaaaggtgctcggacagctccgtggcaagcgggttctgatcaccggtaccactggctttctgggcaaggtggtcctcgaaaggctgattcgggcggtgcctgatatcggcgcaatttacctgctgatccggggcaataaacggcatccggatgctcgttcccgtttcctggaagaaattgccacctcctcggtgtttgaccgtcttcgcgaggccgattcagagggatttgacgcctttctggaagagcgcattcactgcgtgaccggtgaggtgaccgaagcgggtttcgggatagggcaggaagactatcgcaaactcgccaccgaactggatgcggtgatcaactccgctgcaagcgtgaatttccgtgaagagctcgacaaggcgctggccatcaacaccctgtgccttcggaatattgccggcatggtggatttgaatccgaagcttgcggtcctgcaggtctccacctgctatgtcaatggcatgaactcggggcaggtaaccgaatcggtgatcaagccggcaggcgaggccgtgccgcgttccccggacggcttctatgagatagaagagcttgttcgcctgcttcaggataaaattgaagacgttcaggcccgttattccggcaaagtgctggagaggaagctggtggacctggggattcgggaagccaaccgctatggctggagcgatacctacacctttaccaagtggctgggcgaacagttgctgatgaaggcgttaaacgggcgcacgctgaccattctgcgtccttcgattatcgaaagtgccctggaggaaccagcgcccggctggattgagggggtgaaggtggcagatgccatcatcctggcttacgcacgggaaaaagtcaccctcttcccgggcaaacgctccggtatcatcgatgtgattccagtggacctggtggccaactccatcatcctttccctggcggaagctcttggagaacccggtcgacgtcgcatctatcaatgttgcagcgggggcggcaatccaatctccctgggtgagttcatcgatcatctcatggcggaatcaaaagccaattacgctgcctacgatcacctgttctaccggcagcccagcaagccgtttctggcggttaaccgggcgctgtttgatttggtgatcagtggtgttcgcttaccgctctccctgacggaccgtgtgctcaaattactgggaaattcccgggacctgaaaatgctcaggaatctggataccacccagtcgctggcaaccatttttggtttctacaccgcgccggattatatcttccggaacgatgagctgatggcgctggcgaaccggatgggtgaggtcgataaagggctgttcccggtggatgcccgcctgattgactgggagctctacctgcgcaagattcacctggccgggctcaatcgctatgccctgaaagaacgaaaggtgtacagtctgaaaactgcgcgccagcgtaaaaaagctgcctga